(19) United States
(12) Patent Application Publication
(10) Pub. No.: US 2025/0127899 A1
(43) Pub. Date: Apr. 24, 2025

(54) POTASSIUM-SODIUM TARTRATE AS GRANULATING AID

(71) Applicant: BASF SE, Ludwigshafen (DE)

(72) Inventors: Lukas Ries, Ludwigshafen (DE); Thorsten Cech, Ludwigshafen (DE); Elisabeth Gruenstein, Ludwigshafen (DE); Felicitas Guth, Ludwigshafen (DE); Karl Kolter, Limburgerhof (DE)

(21) Appl. No.: 18/727,863

(22) PCT Filed: Feb. 3, 2023
(86) PCT No.: PCT/EP2023/052655

§ 371 Date: Jul. 10, 2024

(30) Foreign Application Priority Data
Feb. 3, 2022 (EP) 22154889.4
Jun. 15, 2022 (EP) 22179206.2
Jan. 26, 2023 (EP) 23153457.9

(51) Int. Cl. A61K 47/12; A61K 9/16

(52) U.S. Cl. CPC A61K 47/12; A61K 9/1617; A61K 9/1682

(57) ABSTRACT

The present invention relates to a powder mixture for the preparation of a granulate comprising as constituent at least a filler or at least an active ingredient or both and as granulation aid potassium sodium tartrate tetrahydrate (PST). Optionally constituents of the powder mixture to be granulated are binders, disintegrants and further customary auxiliaries. It was an object of the present invention to provide a powder mixture which can be granulated without adding a liquid but where the water for the granulation is released in-situ from PST. An additional drying step is not required.

In the area of granulation, especially wet granulation, the addition of water to a raw powder mixture to guarantee particle growth through nucleation always results in a drying step. Drying of the wet granulates is expensive and requires resources such as energy, time, and machine utilization. With the market trend moving from batch processes towards continuous manufacturing this drying step is often performed through fluidized bed-drying, which is on the one hand expensive and on the other hand only a semi-continuous process, because one needs more fluidized bed machines to guarantee the continuity of the process. For a sustainable future a granulate manufactured by a process with reduced drying time and reduced energy to evaporate the water out of the wet granulates is needed. The wet granulation process according to the invention enables a wet granulation of a lot of excipients and active ingredients for which because of their moisture sensitivity the currently used wet granulation process is not suited to be granulated. This advantage in combination with shorter process times will result in a simplification of product development and manufacturing processes and supports the market trend for platform formulations. The platform formulation according to the invention is a powder mixture comprising an excipient that is capable releasing water during the granulation process in combination with a water-binding filler or a water binding active ingredient to reduce or preferably fully eliminate the need to add water as a granulation liquid and would make a drying step obsolete. The present invention relates to platform formulations permitting a granulation by applying thermal energy, such as heat, IR-waves, or microwaves to the powder mixture without adding any liquid. It was shown that Potassium-Sodium tartrate Tetrahydrate (PST) had an outstanding performance as a water releasing excipient enabling granulation of platform formulations (e.g., Na₂tartrate·2 H₂O is not suitable for granulation at all). PST is releasing its water of hydration by the application of the previous described energy input methods. Through this application of energy, it is possible to provide free water for nucleation and profit of the recrystallisation of the salt resulting in dry granulates. It was unexpected that the resulting dry granulates were stable, non-hygroscopic, and very suitable for tableting. The resulting tablets have very high tensile strengths. In further experiments it could be shown that PST can also be used for continuous water-free granulation of active ingredients (e.g., acetyl salicylic acid, paracetamol, ibuprofen). As these active ingredients are known to be difficult to formulate, the results were particularly surprising. PST is also uncritical for oral use, its toxicological daily intake is with 3 g/kg quite high (Kassaian J-M, Ullmann's Encyclopedia Vol. 35, p. 677), and could be used for all solid dosage forms like tablets, pellets, or granules. Also, its long-term toxicology is uncritical.

The performance of PST was particularly surprising, because other potentially water-releasing auxiliaries such as Alum, Calcium acetate, Calcium chloride, carrageenan, corn starch, cyclodextrine, Kaolin, Magnesium acetate, Magnesium citrate, Magnesium sulfate, Sodium citrate, Sodium sulfate, Sodium sulfite, Trisodium phosphate, Sodium phosphate dibasic, Raffinose, rice starch, Sodium acetate, Tragacanth, Trehalose, Tricalcium citrate, wheat starch and Zinc sulfate did not lead to a formation of suitable granulates with the desired increase in particle size, or the properties of the resulting granulates were unfavorable sticky, hygroscopic or showed low compactability and yet others like Sodium sulfate, Magnesium sulfate, Magnesium chloride and dibasic sodium phosphate are indeed technically suitable as a water releasing excipient for granulation purposes but aren't applicable for pharmaceutical dosage forms anyway because of their taste or laxative effect on humans

The present invention relates to a powder mixture for the preparation of a granulate comprising as constituent at least a filler or at least an active ingredient or both and as granulation aid PST. Optionally constituents of the powder mixture to be granulated are binders, disintegrants and further customary auxiliaries. Of course, it is also possible to add the at least one binder and/or at least one disintegrant and/or further customary auxiliaries in dry form to the granulates obtained. Object of the present invention is to provide a powder mixture which can be granulated without adding a liquid but where the water for the granulation is released in situ from PST. An additional dying step is not required. Accordingly, a powder mixture was found comprising a) 10-95% by weight of at least one filler, b) 2.5-10% by weight of at least one binder, c) 2.5-20% by weight of PST, d) 0-7.5% by weight of at least one disintegrant and e) 0-85% by weight of at least one active ingredient the total of the constituents a) to e) being 100% by weight or a powder mixture was found comprising a) 10-95% by weight of at least one filler, b) 2.5-10% by weight of at least one binder, c) 2.5-20% by weight of PST, d) 0-7.5% by weight of at least one disintegrant, e) 0-85% by weight of at least one active ingredient and f) 0-5% by weight further customary auxiliaries the total of the constituents a) to f) being 100% by weight.

The powder mixture comprises as constituent a) from 0 to 98.5% by weight, preferred from 7 to 97% by weight and more preferred from 10 to 95% by weight of a filler. Suitable fillers are, for example, lactose, wherein modified lactose or anhydrous (NF) lactose may be mentioned, starch, in particular modified (pregelatinized) starch, native starch or mixtures of both, calcium phosphate, in particular dibasic, unground dibasic and anhydrous dibasic calcium phosphate, cellulose derivatives, cellulose, in particular microcrystalline cellulose, mannitol, sorbitol, etc. Of course, mixtures of different fillers can be used.

Furthermore, the powder mixture comprises a binder as constituent b) in amounts of from 0 to 15% by weight, preferred from 1.5 to 10% by weight and more preferred from 2.5 to 10% by weight, of the total powder mixture. Suitable binders are water soluble polymers and excipients as well as film forming excipients for example polyvinylpyrrolidones, vinylpyrrolidone/vinylacetate, Copolymers, polyvinyl alcohols, polyvinyl alcohols/polyethylene glycol graft copolymers, polyethylene glycols, ethylene glycol/propylene glycol block copolymers, hydroxypropylmethylcellulose, hydroxypropylcellulose, hydroxyethylcellulose, carrageenans, pectins, xanthans, lactose, sugar alcohols and alginates. Of course, also mixtures of different binders can be used.

Potassium-sodium tartrate tetrahydrate (PST) in amounts of from 1.5 to 30% by weight, preferred 2.5 to 25% by weight and more preferred 2.5 to 20% by weight is used as a granulation aid as constituent c) of the powder mixture. PST with a particle size of about 500 μm d (0.5) 504 μm) can be used as constituent, but it was observed that PST, with a mean particle size d (0.5) of less than 300 μm is particularly suitable as it improves the mixing behavior of the powder mixture.

Furthermore, the powder mixture may comprise as constituent d) disintegrants in amounts of from 0 to 12.5% by weight, preferred from 0 to 10% by weight, more preferred from 0 to 7.5%. Suitable disintegrants are crosslinked polyvinylpyrrolidone, croscarmellose, sodium starch glycolate also meaning according to the invention the sodium and potassium salts thereof. Furthermore, sodium carboxymethylstarch is suitable. Likewise suitable is L-hydroxypropylcellulose, preferably having 5 to 16% hydroxypropoxy groups. Of course, mixtures of different disintegrants can be used.

Optionally the powder mixture may also comprise 0 to 98.5% by weight, preferred from 0 to 90% by weight and more preferred from 0 to 85% of an active ingredient as constituent e) of the powder mixture.

It is possible to employ as active ingredients in principle all active ingredients.

Examples of suitable active ingredients include, but are not limited to: analgesics and antiinflammatory drugs such as fentanyl, indomethacin, ibuprofen, naproxene, diclofenac, diclofenac sodium, fenoprofen, acetylsalicylic acid, ketoprofen, nabumetone, paracetamol, piroxicam, meloxicam, tramadol, and COX-2 inhibitors such as celecoxib and rofecoxib; antiarrhythmic drugs such as procainamide, quinidine and verapamil; antibacterial and antiprotozoal agents such as amoxicillin, ampicillin, benzathine penicillin, benzylpenicillin, cefaclor, cefadroxil, cefprozil, cefuroxime axetil, cephalexin, chloramphenicol, chloroquine, ciprofloxacin, clarithromycin, cla[1]vulanic acid, clindamycin, doxycycline, erythromycin, flucloxacillin sodium, halofantrine, isoniazid, kanamycin sul[1]phate, lincomycin, mefloquine, minocycline, nafcillin sodium, nalidixic acid, neomycin, nortloxacin, ofloxacin, oxa[1]cillin, phenoxymethyl-penicillin potassium, pyrimethaminesulfadoxime and streptomycin; anti-coagulants such as warfarin; antidepressants such as amitriptyline, amoxapine, butriptyline, clomipramine, desipramine, dothiepin, doxepin, fluox[1]etine, reboxetine, amineptine, selegiline, gepirone, imipramine, lithium carbonate, mianserin, milnacipran, nortriptyline, paroxetine, sertraline and 3-[2-[3,4-dihydrobenzofuro[3,2-c]pyridin-2 (1H)-yl]ethyl]-2-methyl-4H-pyrido[1,2-a]py[1]rimidin-4-one; anti-diabetic drugs such as glibenclamide and metformin; anti-epileptic drugs such as carbamazepine, clonazepam, ethosuximide, gabapentin, lamotrigine, levetiracetam, phenobarbitone, phenytoin, primidone, tiagabine, topiramate, valpromide and vigabatrin; antifungal agents such as amphotericin, clotrimazole, econazole, fluconazole, flucytosine, griseofulvin, itraconazole, ketoconazole, miconazole nitrate, nystatin, terbinafine and voriconazole; antihistamines such as astemizole, cinnarizine, cyproheptadine, decarboethoxyloratadine, fexofenadine, flunarizine, levocabastine, loratadine, norastemizole, oxatomide, promethazine and terfenadine; anti-hypertensive drugs such as captopril, enalapril, ketanserin, lisinopril, minoxidil, prazosin, ramipril, reserpine, terazosin and telmisartan; anti-muscarinic agents such as atropine sulphate and hyoscine; antineoplastic agents and antimetabolites such as platinum compounds, such as cisplatin and carboplatin; taxanes such as paclitaxel and docetaxel; tecans such as camptothecin, irinotecan and topotecan; vinca alkaloids such as vinblastine, vindecine, vincristine and vinorelbine; nucleoside derivatives and folic acid antagonists such as 5-fluorouracil, capecitabine, gemcitabine, mercaptopurine, thioguanine, cladribine and methotrexate; alkylating agents such as the nitrogen mustards, e.g. cyclophosphamide, chlorambucil, chlormethine, iphosphamide, melphalan, or the nitrosoureas, e.g. carmustine, lomustine, or other alkylating agents, e.g. busulphan, dacarbazine, procarbazine, thiotepa; antibiotics such as daunorubicin, doxorubicin, idarubicin, epirubicin, bleomycin, dactinomycin and mito[1]mycin; podophyllotoxin derivatives such as etoposide and teniposide; famesyl transferase inhibitors; anthrachinon derivatives such as mitoxantron; anti-migraine drugs such as alniditan, naratriptan and sumatriptan; anti-Parkinsonian drugs such as bromocryptine mesylate, levodopa and selegiline; antipsychotic, hypnotic and sedating agents such as alprazolam, buspirone, chlordiazepoxide, chlorpromazine, clozapine, diazepam, flupenthixol, fluphenazine, flurazepam, 9-hydroxyrisperidone, lorazepam, mazapertine, olan[1]zapine, oxazepam, pimozide, pipamperone, piracetam, promazine, risperidone, selfotel, seroquel, sertindole, sulpir[1]ide, temazepam, thiothixene, triazolam, trifluperidol, ziprasidone and zolpidem; anti-stroke agents such as lubeluzole, lubeluzole oxide, riluzole, aptiganel, eliprodil and remacemide; antitussives such as dextromethorphan and laevodropropizine; antivirals such as acyclovir, ganciclovir, loviride, tivirapine, zidovudine, lamivudine, zidovudine/lamivudine, didano[1]sine, zalcitabine, stavudine, abacavir, lopinavir, amprenavir, nevirapine, efavirenz, delavirdine, indinavir, nelfinavir, ritonavir, saquinavir, adefovir and hydroxyurea; beta-adrenoceptor blocking agents such as atenolol, carvedilol, metoprolol, nebivolol and propanolol; cardiac inotropic agents such as amrinone, digitoxin, digoxin and milrinone; corticosteroids such as beclomethasone dipropionate, betamethasone, budesonide, dexamethasone, hydrocorti[1]sone, methylprednisolone, prednisolone, prednisone and triamcinolone; disinfectants such as chlorhexidine; diuretics such as acetazolamide, furosemide, hydrochlorothiazide and isosorbide; enzymes; gastro-intestinal agents such as cimetidine, cisapride, clebopride, diphenoxylate, domperidone, famotidine, lanso[1]prazole, loperamide, loperamide oxide, mesalazine, metoclopramide, mosapride, nizatidine, norcisapride, olsala[1]zine, omeprazole, pantoprazole, perprazole, prucalopride, rabeprazole, ranitidine, ridogrel and sulphasalazine; haemostatics such as aminocaproic acid; HIV protease inhibiting compounds such as ritonavir, lopinavir, indinavir, saquinavir, tipranavir; lipid regulating agents such as atorvastatin, fenofibrate, fenofibric acid, lovastatin, pravastatin, probucol and simv[1]astatin; local anaesthetics such as benzocaine and lignocaine; opioid analgesics such as buprenorphine, codeine, dextromoramide, dihydrocodeine, hydrocodone, oxycodone and morphine; parasympathomimetics and anti-dementia drugs such as eptastigmine, galanthamine, metrifonate, mil[1]ameline, neostigmine, physostigmine, tacrine, donepezil, rivastigmine, sabcomeline, talsaclidine, xanomeline, me[1]mantine and lazabemide; peptides and proteins such as antibodies, becaplermin, cyclosporine, tacrolimus, erythropoietin, immunoglobulins and insuline; sex hormones such as oestrogens: conjugated oestrogens, ethinyloestradiol, mestranol, oestradiol, oestriol, oestro[1]ne; progestogens; chlormadinone acetate, cyproterone acetate, 17-deacetyl norgestimate, desogestrel, dienogest, dydrogesterone, ethynodiol diacetate, gestodene, 3-keto desogestrel, levonorgestrel, lynestrenol, medroxyprogesterone acetate, megestrol, norethindrone, norethindrone acetate, norethisterone, norethisterone acetate, norethyn[1]odrel, norgestimate, norgestrel, norgestrienone, progesterone and quingestanol acetate; stimulating agents such as sildenafil, vardenafil; vasodilators such as amlodipine, buflomedil, amyl nitrite, diltiazem, dipyridamole, glyceryl trinitrate, isosorbide din[1]itrate, lidoflazine, molsidomine, nicardipine, nifedipine, oxpentifylline and pentaerythritol tetranitrate; their N-oxides, their pharmaceutically acceptable acid or base addition salts, their stereochemically isomeric forms, and their polymorphs Mixtures of active ingredients can also be employed.

Furthermore, the powder mixture may comprise as constituent f) further customary auxiliaries in amounts of from 0 to 15% by weight, preferred from 0 to 10% by weight, more preferred from 0 to 5% by weight. Suitable further customary auxiliaries are selected from flowing agents, acidifying agents, sweeteners, aromas, taste enhancers, thickeners and/or surfactants.

The invention also relates to a process for making granulates from a powder mixture, comprising as constituent at least a filler or at least an active ingredient or both. Whereby the powder mixture to be granulated may also contain binders and/or disintegrants and optionally further customary auxiliaries and the granulation is carried out with mixing by adding a granulation aid, which is potassium-sodium tartrate tetrahydrate (PST), granulating the powder mixture raising the temperature of the mixture to 50° C. or more and inducing the release of at least a part of the crystal water of the PST. Preferred the temperature of the powder mixture is raised to a temperature of between 50° C. and 120° C., most preferred to a temperature of between 80° C. and 110° C. The thermal heat for the temperature enhancement is generated by a heating device. According to the invention all forms of heating devices can be used, preferred are microwave radiation sources, infrared heater, infrared dryer, high shear mixer and extruder, whereby twin-screw extruders are most preferred.

The invention is further illustrated by the following figures and examples

### FIGURES

FIG. 1: High shear screw configuration of the twin-screw extruder used for the granulation

FIG. 2: standard screw configuration of the extruder

### EXAMPLES

### Analytical Methods

Particle size distribution (d (0.1), d (0.5), d (0.9)) of the constituents of the powder mixture and produced granulate was determined using a Malvern Mastersizer 2000 (F.A. Malvern Panalytical). The granules were measured in the dry powder form at 1.0-2.0 bar dispersion air pressure for about 30 to 60 seconds.

The crystal water content (water loss) of PST under the influence of heat was determined

### a) By IR Radiation

Using the heating program of an infrared drying wedge (Sartorius MA 150) 2 g of chemically pure PST were heated to 100° C. The temperature was kept constant till no further mass loss took place. The mass loss allows determination of the crystal water release from PST.

### b) By Ultrasound

For examination of the influence of ultrasonics on crystal water release 3 g chemically pure PST were weighed into a penicillin glass bottle and for one hour sonicated in an ultrasonic bath. The outbound weighing allows then determination of the mass loss.

### c) Microwaves

The impact of microwaves on PST was tested with the help of a microwave oven (Sharp RV10 (750 W; 2450 MHZ). 2 g chemically pure PST were weighed in a watch glass and put for 5 minutes into the microwave oven (power setting 30%). The outbound weighing allows then determination of the mass loss.

### Granulation Using an Extruder

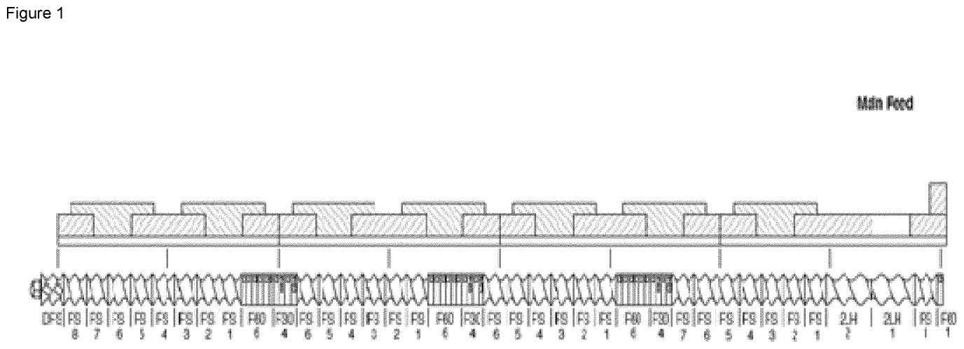

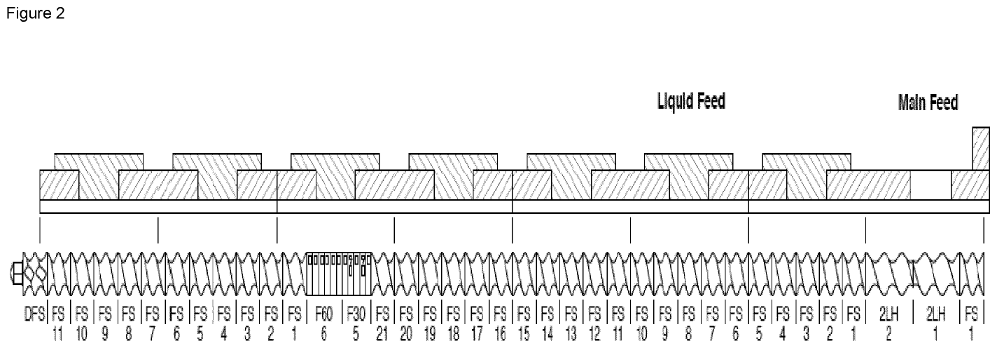

For the granulation of the powder mixture the respective constituents were weighed and shortly mixed by hand. This premix was sieved over 800 μm steel sieve and after that mixed for 10 minutes using a tumble blender, obtaining what is called in the following “Raw material (mixture)”. The Raw material (mixture) was granulated using a twin-screw extruder (Thermo Fisher Scientific, 11 mm) with following parameters (barrel L/D ratio of 40:1) and screw configuration as shown in FIG. 1 or FIG. 2:

| | | Preferred | Most |
|---|---|---|---|
| Parameter | Range | range | preferred range |
| Main process | 25-150° C. | 50-120° C. | 80-110° C. |
| temperature [° C.] | | | |
| Rotation speed [rpm] | 0-600 | 50-250 | 75-150 |
| Throughput [g/h] | 100-1500 | 150-900 | 250-600 |

The granulate leaving the extruder is directly processed further by a milling process or more preferred by a sieving process to display a good uniformity in the particle size distribution to obtain a granulate with a particle size particularly suitable for tableting (see table 9).

Granulation Using a Single Pot Batch Mixer with Jacket Heating (Diosna P1-6)

For the granulation of the powder mixture the respective constituents were weighed and put directly into a 3 L bowl with a jacket heating option. Unless the jacket heating was not needed the trials were conducted at room temperature (RT). The granulation proceeded directly after the powder was put into the granulation machine at the described rotation speed of the crusher and impeller rotor for a particular time range. Eventually a liquid was added during processing. The obtained wet granulates were firstly sieved over 2000 μm steel sieve and eventually dried at room temperature for 72 hours and then sieved over 800 μm steel sieve.

Dry granules gained by the induction of a water releasing and rebinding of the water through various salts may be directly sieved over 800 μm steel sieve.

| | | Preferred | Most |
|---|---|---|---|
| Parameter | Range | range | preferred range |
| Heating jacket | 25-120° C. | 50-100° C. | 70-90° C. |
| temperature [° C.] | | | |
| Rotation speed | 50/500-400/4000 | 100/1000-300/3000 | 150/1500-250/2500 |
| impeller/crusher [rpm] | | | |

### Tableting:

The preparation sieving of the granulates were performed with a vibratory sieve shaker (Retsch—AS 200) within the meshes sizes of 125 μm to 1000 μm for about 2 minutes with an amplitude of 1.0 mm/g.

The sieved fraction between 125 μm to 1000 μm were mixed with additional 1.0% sodium stearyl fumarate in a tumble blender for about 2 minutes. This blend was further used for tableting.

The tableting was conducted in a multifunctional R&D press (Medelpharm—Styl'one Evo) with biplane punches which had different diameters. The compaction pressure varied form 50 MPa to 400 MPa.

The tablets were analyzed (10 tablets per trial) in a semi-automatic tablet hardness tester (Sotax—SmartTest50). The tensile strength can be calculated according to USP <1217>. The disintegration was measured with a disintegration tester (Sotax—DT50) according to USP <701> in distilled water.

TABLE 1
Examples demonstrating which energy input is needed to trigger the water release of PST

| | | | | | | D 4, 3 [μm] |
|---|---|---|---|---|---|---|
| | | | | | | Volume |
| | | | d (0.1) | d (0.5) | d (0.9) | mean |
| trial | Filler/powder mixture | | [μm] | [μm] | [μm] | diameter |
| 1.1 | 100% Granulac 230 | Raw material | 3 | 23 | 59 | 27 |
| 1.2 | 85% Granulac 230, | Raw material | 5 | 31 | 154 | 60 |
| | 10% PST, | (mixture) | | | | |
| | 5% Kollidon 30 | | | | | |
| 1.3 | 85% Granulac 230, | tumble blender | 4 | 27 | 101 | 52 |
| | 10% PST, | (low shear) | | | | |
| | 5% Kollidon 30 | | | | | |
| 1.4 | 85% Granulac 230, | High shearing screw | 3 | 26 | 94 | 43 |
| | 10% PST, | configuration, 30° C. | | | | |
| | 5% Kollidon 30 | | | | | |
| 1.5 | 85% Granulac 230, | High shearing screw | 3 | 25 | 97 | 59 |
| | 10% PST, | configuration, 40° C. | | | | |
| | 5% Kollidon 30 | | | | | |
| 1.6 | 85% Granulac 230, | High shearing screw | 3 | 26 | 122 | 54 |
| | 10% PST, | configuration, 50° C. | | | | |
| | 5% Kollidon 30 | | | | | |
| 1.7 | 85% Granulac 230, | High shearing screw | 45 | 342 | 962 | 433 |
| | 10% PST, | configuration, 80° C. | | | | |
| | 5% Kollidon 30 | | | | | |
| 1.8 | 85% Granulac 230, | High shearing screw | 160 | 604 | 1258 | 664 |
| | 10% PST, | configuration, 100° C. | | | | |
| | 5% Kollidon 30 | | | | | |

¹% means in table 1 and in the following % by weight.

TABLE 2
Examples showing that higher temperatures lead to larger particles and minimum
process temperature is needed for a successful and economic granulation

| | | | | | | D 4, 3 [μm] |
|---|---|---|---|---|---|---|
| | | | | | | Volume |
| | | | d (0.1) | d (0.5) | d (0.9) | mean |
| Trial | powder mixture | | [μm] | [μm] | [μm] | diameter |
| 2.1 | 95% Granulac 230, | Raw material | 3 | 22 | 96 | 40 |
| | 5% Kollicoat IR | (mixture) | | | | |
| 2.2 | 75% Granulac 230, | High shearing screw | 8 | 41 | 242 | 87 |
| | 20% PST, | configuration, 50° C. | | | | |
| | 5% Kollicoat IR | | | | | |
| 2.3 | 85% Granulac 230, | High shearing screw | 49 | 401 | 1211 | 522 |
| | 10% PST, | configuration, 70° C. | | | | |
| | 5% Kollicoat IR | | | | | |
| 2.4 | 85% Granulac 230, | High shearing screw | 63 | 438 | 1308 | 570 |
| | 10% PST, | configuration, 90° C. | | | | |
| | 5% Kollicoat IR | | | | | |
| 2.5 | 85% Granulac 230, | High shearing screw | 92 | 572 | 1370 | 649 |
| | 10% PST, | configuration, 100° C. | | | | |
| | 5% Kollicoat IR | | | | | |
| 2.6 | 85% Granulac 230, | High shearing screw | 173 | 670 | 1407 | 734 |
| | 10% PST, | configuration, 110° C. | | | | |
| | 5% Kollicoat IR | | | | | |

TABLE 3
Water loss of PST under the influence of different energy forms

| | | | | PST | PST | | |
|---|---|---|---|---|---|---|---|
| | | PST (IR | PST (IR | ultrasound | ultrasound | PST | PST |
| Trial | | dryer 1) | dryer 2) | bath 1 | bath 2 | Microwaves 1 | Microwaves 2 |
| 3.1 | Mass before | 2581 | 2926 | 3676.1 | 2461.9 | 5915 | 8685 |
| | treatment [mg] | | | | | | |
| 3.2 | Mass after | 2244 | 2499 | 3670.5 | 2458 | 5403.1 | 7572 |
| | treatment [mg] | | | | | | |
| 3.3 | Mass | 337 | 427 | 5.6 | 3.9 | 511.9 | 1113 |
| | difference [mg] | | | | | | |
| 3.4 | Weight loss [%] | 13.1 | 14.6 | 0.2 | 0.2 | 8.7 | 12.8 |

TABLE 4
Higher amounts of PST in the powder mixture lead to larger particle diameters; needed
temperature for a successful granulation is reduced with growing PST amounts

| | | | | | | | D 4, 3 [μm] |
|---|---|---|---|---|---|---|---|
| | | | | | | | Volume |
| | | | | d (0.1) | d (0.5) | d (0.9) | mean |
| Trial | Filler/powder mixture | | | [μm] | [μm] | [μm] | diameter |
| 4.1 | 100% Granulac 230 | Raw | | 3 | 23 | 59 | 27 |
| | | material | | | | | |
| 4.2 | 95% Granulac 230, | 0% PST | High shearing screw | 3 | 22 | 96 | 40 |
| | 5% Kollicoat IR | | configuration, 100° C. | | | | |
| 4.3 | 92.5% Granulac 230, | 2.5% PST | High shearing screw | 95 | 519 | 1327 | 620 |
| | 5% Kollicoat IR | | configuration, 100° C. | | | | |
| 4.4 | 90% Granulac 230, | 5% PST | High shearing screw | 98 | 551 | 1350 | 639 |
| | 5% Kollicoat IR | | configuration, 100° C. | | | | |
| 4.5 | 85% Granulac 230, | 10% PST | High shearing screw | 92 | 572 | 1370 | 649 |
| | 5% Kollicoat IR | | configuration, 100° C. | | | | |
| 4.6 | 82.5% Granulac 230, | 12.5% PST | High shearing screw | 191 | 628 | 1327 | 701 |
| | 5% Kollicoat IR | | configuration, 100° C. | | | | |
| 4.7 | 80% Granulac 230, | 15% PST | High shearing screw | 222 | 704 | 1391 | 762 |
| | 5% Kollicoat IR | | configuration, 100° C. | | | | |
| 4.8 | 75% Granulac 230, | 20% PST | High shearing screw | 320 | 744 | 1384 | 802 |
| | 5% Kollicoat IR | | configuration, 80° C. | | | | |
| 4.9 | 70% Granulac 230, | 25% PST | High shearing screw | 480 | 907 | 1507 | 951 |
| | 5% Kollicoat IR | | configuration, 100° C. | | | | |

TABLE 5
Na₂-tartrate × 2 H₂O as hydrate in the powder mixture

| | | | | | | D 4, 3 [μm] |
|---|---|---|---|---|---|---|
| | | | | | | Volume |
| | | | d (0.1) | d (0.5) | d (0.9) | mean |
| Trial | powder mix | | [μm] | [μm] | [μm] | diameter |
| 5.1 | 85% Granulac 230, | Raw material | 4 | 27 | 90 | 40 |
| | 10% Na₂-tartrate × 2H₂O, | (mixture) | | | | |
| | 5% Kollidon 30 | | | | | |
| 5.2 | 85% Granulac 230, | High shearing screw | 4 | 30 | 101 | 44 |
| | 10% Na₂-tartrate × 2H₂O, | configuration 100° C. | | | | |
| | 5% Kollidon 30 | | | | | |
| 5.3 | 85% Granulac 230, | High shearing screw | 5 | 31 | 103 | 45 |
| | 10% Na₂-tartrate × 2H₂O, | configuration, 120° C. | | | | |
| | 5% Kollidon 30 | | | | | |

TABLE 6
Different fillers with different amounts of PST performing a successful
granulation under the influence of temperature; references without PST

| | | | | | | | D 4,3 [μm] |
|---|---|---|---|---|---|---|---|
| | | | | | | | Volume |
| | | | | d (0.1) | d (0.5) | d (0.9) | mean |
| Trial | Filler/powder mixture | | | [μm] | [μm] | [μm] | diameter |
| 6.1 | 100% Avicel PH 101 | Raw material | | 22 | 59 | 125 | 68 |
| 6.2 | 95% Avicel PH 101, | 0% PST | screw configuration | 22 | 59 | 135 | 75 |
| | 5% Kollicoat IR | | (FIG. 2), 100° C. | | | | |
| 6.3 | 75% Avicel PH 101, | 20% PST | screw configuration | 34 | 294 | 1143 | 456 |
| | 5% Kollicoat IR, | | (FIG. 2), 100° C. | | | | |
| | 20% PST | | | | | | |
| 6.4 | 100% Avicel PH 105 | Raw material | | 7 | 21 | 48 | 25 |
| 6.5 | 95% Avicel PH 105, | 0% PST | screw configuration | 11 | 36 | 662 | 186 |
| | 5% Kollicoat IR | | (FIG. 2), 100° C. | | | | |
| 6.6 | 85% Avicel PH 105, | 10% PST | screw configuration | 18 | 138 | 878 | 309 |
| | 5% Kollicoat IR, | | (FIG. 2), 100° C. | | | | |
| | 10% PST | | | | | | |
| 6.7 | 100% DICAFOS D14 | Raw material | | 1 | 6 | 27 | 11 |
| 6.8 | 95% DICAFOS 14, | 0% PST | screw configuration | 1 | 9 | 56 | 23 |
| | 5% Kollicoat IR | | (FIG. 2), 100° C. | | | | |
| 6.9 | 85% DICAFOS D14, | 10% PST | screw configuration | 3 | 24 | 297 | 104 |
| | 5% Kollicoat IR, | | (FIG. 2), 100° C. | | | | |
| | 10% PST | | | | | | |
| 6.10 | 100% DICAFOS A12 | Raw material | | | 6 | 30 | |
| 6.11 | 95% DICAFOS A12, | 0% PST | screw configuration | 1 | 13 | 91 | 30 |
| | 5% Kollicoat IR | | (FIG. 2), 100° C. | | | | |
| 6.12 | 85% DICAFOS A12, | 10% PST | screw configuration | 155 | 496 | 1098 | 571 |
| | 5% Kollicoat IR, | | (FIG. 2), 100° C. | | | | |
| | 10% PST | | | | | | |
| 6.13 | 100% DICAFOS A60 | Raw material | | 38 | 64 | 104 | 68 |
| 6.14 | 95% DICAFOS A60, | 0% PST | screw configuration | 33 | 63 | 127 | 101 |
| | 5% Kollicoat IR | | (FIG. 2), 100° C. | | | | |
| 6.15 | 75% DICAFOS A60, | 20% PST | screw configuration | 116 | 278 | 704 | 357 |
| | 5% Kollicoat IR, | | (FIG. 2), 70° C. | | | | |
| | 20% PST | | | | | | |
| 6.16 | 100% Granulac 230 | Raw material | | 3 | 23 | 59 | 27 |
| 6.17 | 95% Granulac 230, | 0% PST | screw configuration | 3 | 22 | 96 | 40 |
| | 5% Kollicoat IR | | (FIG. 2), 95° C. | | | | |
| 6.18 | 85% Granulac 230, | 10% PST | screw configuration | 92 | 572 | 1370 | 649 |
| | 5% Kollicoat IR, | | (FIG. 2), 100° C. | | | | |
| | 10% PST | | | | | | |

TABLE 7
Granulation of powder mixture using different binders

| | | | | | | | D 4, 3 [μm] |
|---|---|---|---|---|---|---|---|
| | | | | | | | Volume |
| | Binder/powder | | | d (0.1) | d (0.5) | d (0.9) | mean |
| Trial | mixture | binder | | [μm] | [μm] | [μm] | diameter |
| 7.1 | 10% PST, | 0% binder | screw configuration | 77 | 176 | 925 | 333 |
| | 90% DICAFOS A60 | | (FIG. 2), 80° C. | | | | |
| 7.2 | 10% PST, | Kollicoat | screw configuration | 59 | 113 | 215 | 135 |
| | 85% DICAFOS A60, | IR | (FIG. 2), 80° C. | | | | |
| | 5% binder | | | | | | |
| 7.3 | 10% PST, | Kollidon | screw configuration | 59 | 112 | 215 | 138 |
| | 85% DICAFOS A60, | 30 | (FIG. 2), 80° C. | | | | |
| | 5% binder | | | | | | |
| 7.4 | 10% PST, | Kollidon | screw configuration | 57 | 111 | 249 | 144 |
| | 85% DICAFOS A60, | 90 | (FIG. 2), 80° C. | | | | |
| | 5% binder | | | | | | |
| 7.5 | 10% PST, | Kollidon | screw configuration | 74 | 154 | 335 | 184 |
| | 85% DICAFOS A60, | VA64 | (FIG. 2), 80° C. | | | | |
| | 5% binder | | | | | | |
| 7.6 | 10% PST, | Soluplus | screw configuration | 77 | 162 | 356 | 198 |
| | 85% DICAFOS A60, | | (FIG. 2), 80° C. | | | | |
| | 5% binder | | | | | | |
| 7.7 | 10% PST, | 0% binder | screw configuration | 109 | 330 | 1083 | 477 |
| | 90% DICAFOS A60 | | (FIG. 2), 100° C. | | | | |
| 7.8 | 10% PST, | Kollicoat | screw configuration | 82 | 169 | 352 | 204 |
| | 85% DICAFOS A60, | IR | (FIG. 2), 100° C. | | | | |
| | 5% binder | | | | | | |
| 7.9 | 10% PST, | Kollidon | screw configuration | 98 | 221 | 634 | 307 |
| | 85% DICAFOS A60, | 30 | (FIG. 2), 100° C. | | | | |
| | 5% binder | | | | | | |
| 7.10 | 10% PST, | Kollidon | screw configuration | 94 | 185 | 352 | 209 |
| | 85% DICAFOS A60, | 90 | (FIG. 2), 100° C. | | | | |
| | 5% binder | | | | | | |
| 7.11 | 10% PST, | Kollidon | screw configuration | 102 | 279 | 725 | 357 |
| | 85% DICAFOS A60, | VA64 | (FIG. 2), 100° C. | | | | |
| | 5% binder | | | | | | |
| 7.12 | 10% PST, | Soluplus | screw configuration | 126 | 333 | 941 | 447 |
| | 85% DICAFOS A60, | | (FIG. 2), 100° C. | | | | |
| | 5% binder | | | | | | |

TABLE 8
Reproducibility

| | | | | | | D 4, 3 |
|---|---|---|---|---|---|---|
| | | | | | | [μm] |
| | | | d | d | d | Volume |
| | | | (0.1) | (0.5) | (0.9) | mean |
| Trial | powder mixture | | [μm] | [μm] | [μm] | diameter |
| 8.1 | 85% Granulac 230, | 5 min | 78 | 459 | 1336 | 593 |
| | 5% Kollicoat IR, | 20 min | 92 | 563 | 1377 | 648 |
| | 10% PST | 40 min | 98 | 594 | 1376 | 666 |
| | | 60 min | 95 | 530 | 1294 | 616 |
| | | 80 min | 99 | 586 | 1369 | 661 |
| | | mean | 92 | 546 | 1350 | 637 |
| 8.2 | 85% Granulac 230, | 5 min | 76 | 477 | 1329 | 596 |
| | 5% Kollicoat IR, | 20 min | 95 | 558 | 1333 | 636 |
| | 10% PST | 40 min | 90 | 482 | 1249 | 582 |
| | | 60 min | 112 | 560 | 1325 | 645 |
| | | 80 min | 123 | 557 | 1318 | 645 |
| | | mean | 99 | 527 | 1311 | 621 |

TABLE 9
Comparison of granulates directly warm sieved after extrusion with unsieved granulates

| | | | | | | | D 4, 3 [μm] |
|---|---|---|---|---|---|---|---|
| | | | | | | | Volume |
| | Binder/powder | | | d (0.1) | d (0.5) | d (0.9) | mean |
| Trial | mixture | | | [μm] | [μm] | [μm] | diameter |
| 9.1 | 85% Granulac 230, | Before | screw configuration | 112 | 504 | 1250 | 600 |
| | 10% PST, | sieving | (FIG. 2), 100° C. | | | | |
| | 5% Kollidon 30 | | | | | | |
| 9.2 | 85% Granulac 230, | After | screw configuration | 63 | 274 | 722 | 342 |
| | 10% PST, | sieving | (FIG. 2), 100° C. | | | | |
| | 5% Kollidon 30 | | | | | | |

TABLE 10
Granulation of powder mixtures containing a disintegrant

| | | | | | | | D 4,3 [μm] |
|---|---|---|---|---|---|---|---|
| | | | | | | | Volume |
| | | | | d (0.1) | d (0.5) | d (0.9) | mean |
| Trial | Powder mixture | | | [μm] | [μm] | [μm] | diameter |
| 10.1 | 72% Granulac 230, | Raw material | | 4 | 31 | 170 | 78 |
| | 10% PST, | (mixture) | | | | | |
| | 5% Kollidon 30, | | | | | | |
| | 3% Kollidon CL, | | | | | | |
| | 10% Propranolol HCl | | | | | | |
| 10.2 | 10% Propranolol HCl, | 0% disintegrant | screw configuration | 44 | 188 | 1086 | 412 |
| | 75% Granulac 230, | | (FIG. 2), 100° C. | | | | |
| | 5% Kollidon K30 | | | | | | |
| 10.3 | 72% Granulac 230, | 10% propranolol + | screw configuration | 72 | 350 | 1105 | 476 |
| | 10% PST, | 3% disintegrant | (FIG. 2), 100° C. | | | | |
| | 5% Kollidon 30, | | | | | | |
| | 3% Kollidon CL, | | | | | | |
| | 10% Propranolol HCl | | | | | | |

TABLE 11
Tableted granulates according to table 7 & 10

| | compression | tensile | | | | ejection | disintegration |
|---|---|---|---|---|---|---|---|
| | pressure | strength | mass | thickness | diameter | force | time |
| | [MPa] | [N/mm2] | [mg] | [mm] | [mm] | [N] | [s] |
| Granulation | 59.9 | 0.4 | 228.0 | 3.8 | 8.0 | 290 | |
| trial 10.2 | 116.2 | 1.1 | 231.3 | 3.6 | 8.0 | 542 | |
| tableted | 172.3 | 1.8 | 231.0 | 3.4 | 8.0 | 648 | 403 |
| | 225.0 | 2.5 | 234.0 | 3.4 | 8.0 | 782 | |
| | compression | tensile | | | | ejection | disintegration |
| | pressure | strength | mass | thickness | diameter | force | time |
| | [MPa] | [N/mm2] | [mg] | [mm] | [mm] | [N] | [s] |
| Granulation | 59.7 | 1.1 | 235.2 | 4.1 | 8.0 | 103 | |
| trial 10.3 | 118.6 | 3.1 | 236.9 | 3.7 | 8.1 | 135 | |
| tableted | 177.7 | 5.0 | 238.6 | 3.5 | 8.0 | 149 | 250 |
| | 235.6 | 6.1 | 239.6 | 3.4 | 8.1 | 162 | |
| | compression | tensile | | | | ejection | disintegration |
| | pressure | strength | mass | thickness | diameter | force | time |
| | [MPa] | [N/mm2] | [mg] | [mm] | [mm] | [N] | [s] |
| Granulation | 93.9 | 0.4 | 402.8 | 4.2 | 8.0 | 94 | |
| trial 7.7 | 185.8 | 1.4 | 407.1 | 3.8 | 8.0 | 186 | |
| tableted | 294.2 | 3.0 | 383.2 | 3.4 | 8.0 | 294 | |
| | 364.7 | 4.6 | 356.7 | 3 | 8.0 | 364 | |
| | compression | tensile | | | | ejection | disintegration |
| | pressure | strength | mass | thickness | diameter | force | time |
| | [MPa] | [N/mm2] | [mg] | [mm] | [mm] | [N] | [s] |
| Granulation | 93.5 | 0.7 | 416.5 | 4.7 | 8.3 | 94 | |
| trial 7.8 | 187 | 2.8 | 426.6 | 4.2 | 8.1 | 187 | |
| tableted | 275.7 | 5.3 | 401.4 | 3.6 | 8.1 | 276 | |
| | 364.1 | 6.7 | 389.6 | 3.4 | 8.1 | 364 | |
| | compression | tensile | | | | ejection | disintegration |
| | pressure | strength | mass | thickness | diameter | force | time |
| | [MPa] | [N/mm2] | [mg] | [mm] | [mm] | [N] | [s] |
| Granulation | 92.3 | 1.4 | 401.9 | 4.3 | 8.0 | 92 | |
| trial 7.9 | 184.4 | 4.4 | 397.6 | 3.7 | 8.0 | 184 | |
| tableted | 275.1 | 7.7 | 379.3 | 3.3 | 8.0 | 275 | |
| | 366.7 | 10.3 | 353.0 | 3.0 | 8.0 | 367 | |
| | compression | tensile | | | | ejection | disintegration |
| | pressure | strength | mass | thickness | diameter | force | time |
| | [MPa] | [N/mm2] | [mg] | [mm] | [mm] | [N] | [s] |
| Granulation | 91.7 | 1.8 | 406.0 | 4.5 | 8.0 | 92 | |
| trial 7.10 | 184.6 | 5.2 | 413.6 | 4.0 | 8.0 | 185 | |
| tableted | 275.9 | 7.7 | 411.2 | 3.8 | 8.0 | 276 | |
| | 362.9 | 9.8 | 403.8 | 3.6 | 8.0 | 363 | |
| | compression | tensile | | | | ejection | disintegration |
| | pressure | strength | mass | thickness | diameter | force | time |
| | [MPa] | [N/mm2] | [mg] | [mm] | [mm] | [N] | [s] |
| Granulation | 91.9 | 1.2 | 428.6 | 4.4 | 8.0 | 92 | |
| trial 7.12 | 183.8 | 3.2 | 419.5 | 3.9 | 8.0 | 184 | |
| tableted | 271.8 | 5.4 | 420.9 | 3.7 | 8.0 | 272 | |
| | 359.1 | 6.6 | 389.0 | 3.3 | 8.0 | 359 | |

TABLE 12
Granulation of powder mixtures containing different APIs using PST as granulation aid

| | | | | | | D 4, 3 [μm] |
|---|---|---|---|---|---|---|
| | | | | | | Volume |
| | | | d (0.1) | d (0.5) | d (0.9) | mean |
| Trial | Powder mixture | | [μm] | [μm] | [μm] | diameter |
| 12.1 | 100% Propranolol HCl | Raw material | 2 | 11 | 50 | 20 |
| 12.2 | 10% Propranolol HCl, | Raw material | 3 | 26 | 113 | 58 |
| | 10% PST, | (mixture) | | | | |
| | 75% Granulac 230, | | | | | |
| | 5% Kollidon 30 | | | | | |
| 12.3 | 10% Propranolol HCl, | screw configuration | 44 | 188 | 1086 | 412 |
| | 10% PST, | (FIG. 2), 100° C. | | | | |
| | 75% Granulac 230, | | | | | |
| | 5% Kollidon 30 | | | | | |
| 12.4 | 99.5% Ibuprofen 50, | Raw material | 3 | 19 | 65 | 29 |
| | 0.5% Aerosil 200 | (mixture) | | | | |
| 12.5 | 69.5% Ibuprofen 50, | Raw material | 4 | 31 | 130 | 55 |
| | 10% PST, | (mixture) | | | | |
| | 15% Granulac 230, | | | | | |
| | 5% Kollidon 30, | | | | | |
| | 0.5% Aerosil 200 | | | | | |
| 12.6 | 69% Ibuprofen 50, | screw configuration | 178 | 587 | 1247 | 657 |
| | 10% PST, | (FIG. 2), 55° C. | | | | |
| | 15% Granulac 230, | | | | | |
| | 5% Kollidon 30, | | | | | |
| | 1% Aerosil | | | | | |
| 12.7 | 100% Acetylsalicylic | Raw material | 4 | 28 | 120 | 47 |
| | acid fine (ASA) | | | | | |
| 12.8 | 75% ASA, 10% PST, | Raw material | 5 | 31 | 154 | 60 |
| | 10% Granulac 230, | (mixture) | | | | |
| | 5% Kollidon 30 | | | | | |
| 12.9 | 75% ASA, 10% PST, | screw configuration | 278 | 593 | 1121 | 654 |
| | 10% Granulac 230, | (FIG. 2), 80° C. | | | | |
| | 5% Kollidon 30 | | | | | |

TABLE 13
Granulation of different salts incl. water of hydration
in a batch single pot mixer with jacked heating

| | | | | | | D 4, 3 [μm] |
|---|---|---|---|---|---|---|
| | | | | | | Volume |
| | | | d (0.1) | d (0.5) | d (0.9) | mean |
| Trial | Powder mixture | | [μm] | [μm] | [μm] | diameter |
| 13.1 | 100% Granulac 230 | Raw material | 3 | 23 | 59 | 27 |
| 13.2 | 70% Granulac 230, | 67° C. process | 123 | 220 | 433 | 275 |
| | 5% Kollicoat IR, | temperature, | | | | |
| | 20% PST | Impeller/crusher | | | | |
| | | 400/2000 rpm | | | | |
| 13.3 | 75% Granulac 230, | 65° C. process | 124 | 405 | 1165 | 536 |
| | 5% Kollicoat IR, | temperature, | | | | |
| | 20% Na₂HPO₄ × 12 H₂O | Impeller/crusher | | | | |
| | | 400/2000 rpm | | | | |
| 13.4 | 70% Granulac 230, | 85° C. process | 37 | 193 | 609 | 270 |
| | 5% Kollicoat IR, | temperature, | | | | |
| | 25% MgSO₄ × 7 H₂O | Impeller/crusher | | | | |
| | | 400/2000 rpm | | | | |
| 13.5 | 75% Granulac 230, | 40° C. process | 50 | 191 | 506 | 239 |
| | 5% Kollicoat IR, | temperature, | | | | |
| | 20% NaSO₄ × 10 H₂O | Impeller/crusher | | | | |
| | | 400/2000 rpm | | | | |
| 13.6 | 70% Granulac 230, | 75° C. process | 109 | 377 | 743 | 404 |
| | 5% Kollicoat IR, | temperature, | | | | |
| | 25% MgCl₂ × 6 H₂O | Impeller/crusher | | | | |
| | | 400/2000 rpm | | | | |

TABLE 14
Granulation of powder mixtures containing Paracetamol using PST as granulation aid

| | | | | | | D (4, 3) [μm] - |
|---|---|---|---|---|---|---|
| | | | | | | Volume |
| | | | d(0.1) | d(0.5) | d(0.9) | weighted |
| Trial | Powder mixture | | [μm] | [μm] | [μm] | mean |
| 14.1 | 99% Paracetamol, | raw material | 2 | 9 | 40 | 16 |
| | 1% Aerosil | | | | | |
| 14.2 | 84% Paracetamol, | screw | 13 | 140 | 1108 | 384 |
| | 15% PST, | configuration, | | | | |
| | 1% Aerosil | (FIG. 2), 100° C. | | | | |
| 14.3 | 79% Paracetamol, | screw | 101 | 483 | 1188 | 572 |
| | 15% PST, | configuration, | | | | |
| | 5% Kollidon 30, | (FIG. 2), 100° C. | | | | |
| | 1% Aerosil | | | | | |
| 14.4 | 84% Paracetamol, | screw | 22 | 185 | 823 | 318 |
| | 10% PST, | configuration, | | | | |
| | 5% Kollidon 30, | (FIG. 2), 100° C. | | | | |
| | 1% Aerosil | | | | | |
| 14.5 | 84% Paracetamol, | screw | 153 | 627 | 1290 | 681 |
| | 10% PST, | configuration, | | | | |
| | 5% Kollidon 30, | (FIG. 2), 120° C. | | | | |
| | 1% Aerosil | | | | | |
| 14.6 | 74% Paracetamol, | screw | 14 | 132 | 1060 | 361 |
| | 15% PST, | configuration, | | | | |
| | 10% Starch 1500, | (FIG. 2), 100° C. | | | | |
| | 1% Aerosil | | | | | |
| 14.7 | 80% Paracetamol, | screw | 12 | 80 | 528 | 186 |
| | 10% PST, | configuration, | | | | |
| | 5% Kollidon 30, | (FIG. 2), 100° C. | | | | |
| | 4% Kollidon CL-F, | | | | | |
| | 1% Aerosil | | | | | |
| 14.8 | 80% Paracetamol, | screw | 10 | 80 | 561 | 197 |
| | 10% PST, | configuration, | | | | |
| | 5% Kollidon 30, | (FIG. 2), 100° C. | | | | |
| | 4% Kollidon CL, | | | | | |
| | 1% Aerosil | | | | | |
| 14.9 | 75% Paracetamol, | screw | 62 | 371 | 1041 | 475 |
| | 15% PST, | configuration, | | | | |
| | 5% Kollidon 30, | (FIG. 2), 100° C. | | | | |
| | 4% Kollidon CL, | | | | | |
| | 1% Aerosil | | | | | |
| 14.10 | 75% Paracetamol, | screw | 22 | 162 | 862 | 316 |
| | 15% PST, | configuration, | | | | |
| | 5% Kollidon 30, | (FIG. 2), 100° C. | | | | |
| | 4% Kollidon CL-F, | | | | | |
| | 1% Aerosil | | | | | |
| 14.11 | 71% Paracetamol, | screw | 14 | 103 | 525 | 195 |
| | 15% PST, | configuration, | | | | |
| | 5% Kollidon 30, | (FIG. 2), 100° C. | | | | |
| | 8% Kollidon CL, | | | | | |
| | 1% Aerosil | | | | | |
| 14.12 | 75% Paracetamol, | screw | 104 | 594 | 991 | 584 |
| | 15% PST, | configuration, | | | | |
| | 5% Kollidon 30, | (FIG. 2), 100° C. | | | | |
| | 4% Sodium starch | | | | | |
| | glycolate, | | | | | |
| | 1% Aerosil | | | | | |
| 14.13 | 75% Paracetamol, | screw | 73 | 470 | 917 | 476 |
| | 15% PST, | configuration, | | | | |
| | 5% Kollidon 30, | (FIG. 2), 100° C. | | | | |
| | 4% Croscramellose | | | | | |
| | sodium, 1% Aerosil | | | | | |
| 14.14 | 75% Paracetamol, | screw | 67 | 523 | 977 | 520 |
| | 15% PST, | configuration, | | | | |
| | 5% Kollidon 30, | (FIG. 2), 100° C. | | | | |
| | (4% Kollidon CL | | | | | |
| | added after | | | | | |
| | granulation before | | | | | |
| | tableting), | | | | | |
| | 1% Aerosil | | | | | |
| 14.15 | 74% Paracetamol, | screw | 100 | 592 | 1352 | 663 |
| | 10% PST, | configuration, | | | | |
| | 10% Granulac 230, | (FIG. 2), 100° C. | | | | |
| | 5% Kollidon 30, | | | | | |
| | 1% Aerosil | | | | | |
| 14.16 | 90% Paracetamol, | single pot batch | 22 | 249 | 1112 | 422 |
| | 5% Kollidon 30, | wet granulation, | | | | |
| | 4% Kollidon CL, | room temperature, | | | | |
| | 1% Aerosil | Impeller/crusher | | | | |
| | | 500/2200 rpm | | | | |
| 14.17 | 90% Paracetamol, | single pot batch | 35 | 220 | 1179 | 441 |
| | 5% Kollidon 30, | wet granulation, | | | | |
| | 4% Sodium starch | room temperature, | | | | |
| | glycolate, | Impeller/crusher | | | | |
| | 1% Aerosil | 500/2200 rpm | | | | |
| 14.18 | 90% Paracetamol, | single pot batch | 41 | 194 | 1101 | 404 |
| | 5% Kollidon 30, | wet granulation, | | | | |
| | 4% Croscramellose | room temperature, | | | | |
| | sodium, 1% Aerosil | Impeller/crusher | | | | |
| | | 500/2200 rpm | | | | |
| 14.19 | 84% Paracetamol, | single pot batch | 41 | 184 | 590 | 266 |
| | 10% Granulac 230, | wet granulation, | | | | |
| | 5% Kollidon 30 | room temperature, | | | | |
| | | Impeller/crusher | | | | |
| | | 500/2200 rpm | | | | |

TABLE 15
Paracetamol tablets manufactured by granulates according to the invention
and granulates manufactured according to a batch granulation process

| | compression | tensile | | | | ejection |
|---|---|---|---|---|---|---|
| | pressure | strength | mass | thickness | diameter | force |
| | [Mpa] | [N/mm²] | [mg] | [mm] | [mm] | [N] |
| Granulation | 58.7 | 1.1 | 593.3 | 4.7 | 12.1 | 137 |
| trial 14.2 | 122.3 | 2.3 | 592.6 | 4.3 | 12.1 | 219 |
| tableted | 173.7 | 3 | 587.8 | 4.2 | 12.1 | 271 |
| | 229.1 | 3.4 | 588 | 4.1 | 12.1 | 290 |
| | compression | tensile | | | | ejection |
| | pressure | strength | mass | thickness | diameter | force |
| | [Mpa] | [N/mm²] | [mg] | [mm] | [mm] | [N] |
| Granulation | 58.7 | 1.5 | 635.1 | 5.1 | 12.1 | 191 |
| trial 14.3 | 117.6 | 3.4 | 628 | 4.5 | 12.1 | 249 |
| tableted | 175.7 | 4.6 | 622.6 | 4.4 | 12.1 | 253 |
| | 231 | 5.4 | 601.4 | 4.1 | 12.1 | 244 |
| | compression | tensile | | | | ejection |
| | pressure | strength | mass | thickness | diameter | force |
| | [Mpa] | [N/mm²] | [mg] | [mm] | [mm] | [N] |
| Granulation | 57.9 | 0.9 | 598.8 | 5.1 | 12.1 | 341 |
| trial 14.4 | 120.4 | 2.1 | 602.4 | 4.7 | 12.1 | 582 |
| tableted | 168.6 | 2.9 | 606.1 | 4.5 | 12.1 | 714 |
| | 225 | 4.2 | 579.4 | 4.1 | 12.1 | 686 |
| | compression | tensile | | | | ejection |
| | pressure | strength | mass | thickness | diameter | force |
| | [Mpa] | [N/mm²] | [mg] | [mm] | [mm] | [N] |
| Granulation | 56.8 | 0.7 | 589.5 | 5.1 | 12.1 | 458 |
| trial 14.5 | 112.4 | 2.1 | 583.6 | 4.5 | 12.1 | 683 |
| tableted | 168.6 | 3.3 | 568.2 | 4.2 | 12.1 | 775 |
| | 222.2 | 4.1 | 553.9 | 4 | 12.1 | 829 |
| | compression | tensile | | | | ejection |
| | pressure | strength | mass | thickness | diameter | force |
| | [Mpa] | [N/mm²] | [mg] | [mm] | [mm] | [N] |
| Granulation | 60 | 1.1 | 664.9 | 5 | 12.1 | 115 |
| trial 14.6 | 118.7 | 1.9 | 667.1 | 4.7 | 12.1 | 155 |
| tableted | 175.2 | 2.3 | 667.5 | 4.7 | 12.1 | 149 |
| | 228.2 | 2.4 | 670.4 | 4.7 | 12.1 | 138 |
| | compression | tensile | | | | ejection |
| | pressure | strength | mass | thickness | diameter | force |
| | [Mpa] | [N/mm²] | [mg] | [mm] | [mm] | [N] |
| Granulation | 56.4 | 0.5 | 619.4 | 5.4 | 12.1 | 508 |
| trial 14.7 | 110.4 | 1.4 | 628.7 | 5 | 12.1 | 1185 |
| tableted | 163 | 2.3 | 630.9 | 4.8 | 12.1 | 1674 |
| | 217.5 | 3 | 621.1 | 4.6 | 12.1 | 1795 |
| | compression | tensile | | | | ejection |
| | pressure | strength | mass | thickness | diameter | force |
| | [Mpa] | [N/mm²] | [mg] | [mm] | [mm] | [N] |
| Granulation | 58.4 | 0.4 | 641.1 | 5.6 | 12.1 | 297 |
| trial 14.8 | 115.4 | 1.2 | 621.1 | 5 | 12.1 | 446 |
| tableted | 171.9 | 2 | 610.4 | 4.7 | 12.1 | 541 |
| | 227.9 | 2.7 | 598.9 | 4.5 | 12.1 | 560 |
| | compression | tensile | | | | ejection |
| | pressure | strength | mass | thickness | diameter | force |
| | [Mpa] | [N/mm²] | [mg] | [mm] | [mm] | [N] |
| Granulation | 56.2 | 0.9 | 672 | 5.6 | 12.1 | 182 |
| trial 14.9 | 113 | 2.5 | 669.9 | 5 | 12.1 | 255 |
| tableted | 168.7 | 3.7 | 660 | 4.7 | 12.1 | 285 |
| | 225.8 | 4.3 | 635.2 | 4.5 | 12.1 | 275 |
| | compression | tensile | | | | ejection |
| | pressure | strength | mass | thickness | diameter | force |
| | [Mpa] | [N/mm²] | [mg] | [mm] | [mm] | [N] |
| Granulation | 56.1 | 0.9 | 662 | 5.5 | 12.1 | 199 |
| trial 14.10 | 82.5 | 1.7 | 635.1 | 5 | 12.1 | 236 |
| tableted | 112.9 | 2.6 | 665.1 | 5 | 12.1 | 277 |
| | 168.7 | 3.6 | 662.2 | 4.7 | 12.1 | 298 |
| | 223.7 | 4 | 659.9 | 4.6 | 12.1 | 307 |
| | compression | tensile | | | | ejection |
| | pressure | strength | mass | thickness | diameter | force |
| | [Mpa] | [N/mm²] | [mg] | [mm] | [mm] | [N] |
| Granulation | 54 | 0.6 | 698.7 | 6.1 | 12.1 | 446 |
| trial 14.11 | 108 | 1.8 | 697.3 | 5.4 | 12.1 | 397 |
| tableted | 163.6 | 2.9 | 694.4 | 5.1 | 12 | 338 |
| | 217.5 | 3.4 | 690.1 | 5 | 12 | 321 |
| | compression | tensile | | | | ejection |
| | pressure | strength | mass | thickness | diameter | force |
| | [Mpa] | [N/mm²] | [mg] | [mm] | [mm] | [N] |
| Granulation | 57.4 | 1.4 | 648.7 | 5.1 | 12.1 | 164 |
| trial 14.12 | 114.4 | 3.1 | 651.8 | 4.7 | 12.1 | 195 |
| tableted | 170 | 4.3 | 652.5 | 4.5 | 12.1 | 209 |
| | 224.9 | 4.8 | 648.7 | 4.4 | 12.1 | 214 |
| | compression | tensile | | | | ejection |
| | pressure | strength | mass | thickness | diameter | force |
| | [Mpa] | [N/mm²] | [mg] | [mm] | [mm] | [N] |
| Granulation | 57.2 | 1.4 | 652.1 | 5.2 | 12.1 | 170 |
| trial 14.13 | 114.1 | 3 | 654.2 | 4.7 | 12 | 207 |
| tableted | 169.8 | 4.4 | 655.4 | 4.6 | 12 | 222 |
| | 223.8 | 5 | 658 | 4.5 | 12.1 | 223 |
| | compression | tensile | | | | ejection |
| | pressure | strength | mass | thickness | diameter | force |
| | [Mpa] | [N/mm²] | [mg] | [mm] | [mm] | [N] |
| Granulation | 56.9 | 1 | 661.5 | 5.6 | 12.1 | 191 |
| trial 14.14 | 113.6 | 2.7 | 661.1 | 5 | 12.1 | 249 |
| tableted | 168.8 | 3.9 | 664.5 | 4.8 | 12.1 | 258 |
| | 224.1 | 4.7 | 664.8 | 4.7 | 12.1 | 261 |
| | compression | tensile | | | | ejection |
| | pressure | strength | mass | thickness | diameter | force |
| | [Mpa] | [N/mm²] | [mg] | [mm] | [mm] | [N] |
| Granulation | 40.6 | 1 | 668.7 | 5.4 | 12.1 | 183 |
| trial 14.15 | 81.9 | 2.2 | 667.5 | 4.9 | 12.1 | 313 |
| tableted | 121.5 | 3.2 | 665.5 | 4.7 | 12.1 | 357 |
| | 158.9 | 3.7 | 654.6 | 4.5 | 12.1 | 431 |
| | compression | tensile | | | | ejection |
| | pressure | strength | mass | thickness | diameter | force |
| | [Mpa] | [N/mm²] | [mg] | [mm] | [mm] | [N] |
| Granulation | 59.9 | 0.5 | 569.2 | 4.9 | 12.1 | 239 |
| trial 14.16 | 118 | 1.4 | 573.8 | 4.6 | 12.1 | 389 |
| tableted | 175 | 2.2 | 575 | 4.4 | 12.1 | 458 |
| | 230.7 | 2.8 | 575 | 4.3 | 12.1 | 473 |
| | compression | tensile | | | | ejection |
| | pressure | strength | mass | thickness | diameter | force |
| | [Mpa] | [N/mm²] | [mg] | [mm] | [mm] | [N] |
| Granulation | 58.5 | 0.5 | 562.8 | 4.8 | 12.1 | 287 |
| trial 14.17 | 115.7 | 1.2 | 569.9 | 4.5 | 12.1 | 486 |
| tableted | 170.9 | 1.9 | 560 | 4.2 | 12.1 | 598 |
| | 227 | 2.5 | 563.6 | 4.2 | 12.1 | 631 |
| | compression | tensile | | | | ejection |
| | pressure | strength | mass | thickness | diameter | force |
| | [Mpa] | [N/mm²] | [mg] | [mm] | [mm] | [N] |
| Granulation | 58.2 | 0.4 | 565.6 | 5 | 12.1 | 284 |
| trial 14.18 | 114.3 | 1 | 570.9 | 4.6 | 12.1 | 481 |
| tableted | 170.6 | 1.7 | 560.9 | 4.3 | 12.1 | 563 |
| | 226.5 | 2.3 | 563.4 | 4.2 | 12.1 | 609 |
| | compression | tensile | | | | ejection |
| | pressure | strength | mass | thickness | diameter | force |
| | [Mpa] | [N/mm²] | [mg] | [mm] | [mm] | [N] |
| Granulation | 38.4 | 0.4 | 632.5 | 5.5 | 12.2 | 313 |
| trial 14.19 | 76.1 | 0.9 | 637.8 | 5.1 | 12.1 | 230 |
| tableted | 114.6 | 1.5 | 636.8 | 4.9 | 12.1 | 637 |
| | 163.5 | 2.2 | 635.5 | 4.7 | 12.1 | 741 |

1. A powder mixture for making granulates comprising:
a. 0-98.5% by weight of at least one filler,
b. 0-15% by weight of at least one binder,
c. 1.5-30% by weight of potassium-sodium tartrate tetrahydrate (PST),
d. 0-12.5% by weight of at least one disintegrant
e. 0-98.5% by weight of at least one active ingredient
a total of constituents a. to e being 100% by weight.
or
a powder mixture for making granulates comprising
a. 0-98.5% by weight of at least one filler,
b. 0-15% by weight of at least one binder,
c. 1.5-30% by weight of PST,
d. 0-12.5% by weight of at least one disintegrant
e. 0-98.5% by weight of at least one active ingredient
f. 0-15% by weight further customary auxiliaries
a total of constituents a. to f being 100% by weight.

2. A powder mixture for making granulates comprising:
a. 7-97% by weight of at least one filler,
b. 1.5-10% by weight of at least one binder,
c. 1.5-25% by weight of PST,
d. 0-10% by weight of at least one disintegrant
e. 0-90% by weight of at least one active ingredient
a total of constituents a. to e being 100% by weight
or
a powder mixture for making granulates comprising
a. 7-97% by weight of at least one filler,
b. 1.5-10% by weight of at least one binder,
c. 1.5-25% by weight of PST,
d. 0-10% by weight of at least one disintegrant
e. 0-98.5% by weight of at least one active ingredient
f. 0-10% by weight further customary auxiliaries
a total of constituents a. to f being 100% by weight.

3. A powder mixture for making granulates comprising:
a. 10-95% by weight of at least one filler,
b. 2.5-10% by weight of at least one binder,
c. 2.5-20% by weight of PST,
d. 0-7.5% by weight of at least one disintegrant
e. 0-85% by weight of at least one active ingredient
a total of constituents a. to e being 100% by weight
or
a powder mixture for making granulates comprising
a. 10-95% by weight of at least one filler,
b. 2.5-15% by weight of at least one binder,
c. 2.5-20% by weight of PST,
d. 0-7.5% by weight of at least one disintegrant
e. 0-85% by weight of at least one active ingredient
f. 0-15% by weight further customary auxiliaries
a total of constituents a. to f being 100% by weight.

4. The powder mixture according to claim 1, wherein a mean particle size d(0.5) of said PST particles is less than 300 μm.

5. A process for making granulates from a powder mixture comprising at least a filler or at least an active ingredient or both and optionally at least one binder and/or optionally at least one disintegrant and/or optionally at least one further customary auxiliary, comprising carrying out the granulation with mixing by adding a granulation aid, which is potassium-sodium tartrate tetrahydrate (PST), granulating the powder mixture by raising a temperature of the powder mixture to 50° C. or more and inducing the release of at least a part of the crystal water of the PST.

6. The process according to claim 5, wherein the temperature of the powder mixture to be granulated is raised to between 8° and 110° C.

7. The process according to claim 5, wherein the temperature of the powder mixture is raised by a heating device.

8. The process according to claim 7, wherein the heating device is a microwave radiation source or an IR-Dryer or an IR-heater or a high shear mixer or an extruder or a combination thereof

9. The process according to claim 8, wherein the heating device is an extruder.

10. The process according to claim 5, wherein the granulates produced are directly milled or sieved.

11. The process according to claim 5, wherein the granulates are produced continuously.

12. A granulate obtained by a process according to claim 5 for tableting or filling capsules.

13. A granulation aid comprising PST as a constituent in a powder mixture.
